(19) United States
(12) Patent Application Publication
(10) Pub. No.: US 2014/0274864 A1
(43) Pub. Date: Sep. 18, 2014

(54) Process of Forming a Personal Care Article

(71) Applicant: The Procter & Gamble Company, Cincinnati, OH (US)

(72) Inventors: Robert Wayne GLENN, JR., Liberty Twp, OH (US); Todd Ryan THOMPSON, Loveland, OH (US); Martin Ian JAMES, Cincinnati, OH (US)

(73) Assignee: The Procter & Gamble Company, Cincinnati, OH (US)

(21) Appl. No.: 14/212,308

(22) Filed: Mar. 14, 2014

Related U.S. Application Data
(60) Provisional application No. 61/794,985, filed on Mar. 15, 2013.

(51) Int. Cl. A61K 8/02; A61Q 19/10; A61Q 5/02

(52) U.S. Cl. CPC A61K 8/0216; A61Q 5/02; A61Q 19/10
USPC 510/403

(57) ABSTRACT

A process for forming a personal care article, the process including (a) adding one or more water soluble polymers and one or more plasticizers to a kneader to form a premix; (b) adding one or more anionic surfactants to the premix to form a mixture; (c) kneading the mixture until homogeneous; and (d) forming the personal care article. The personal care article includes (i) from about 10% to about 60% of one or more anionic surfactants; (ii) from about 10% to about 50% of one or more water soluble polymers; (iii) from about 1% to about 30% of one or more plasticizers; and (iv) from about 10% to about 50% water. The one or more anionic surfactants have a Krafft point of less than about 30° C.

### FIELD OF THE INVENTION

The present invention relates to a process of forming a personal care article using a kneader comprising a surfactant, a water soluble polymer, a plasticizer, and water.

### BACKGROUND OF THE INVENTION

Solid soaps are generally harsh and lead to a squeaky feel on the skin and hair. These qualities are generally unacceptable for many of today's consumers. Anionic surfactants such as alkyl ether sulfates have been developed to improve upon the disadvantages of solid soaps. However, many anionic surfactants have low Krafft points and are thereby generally formulated only in liquid products. This is one of the primary reasons for the proliferation of liquid shampoos and liquid body washes across the personal care industry. While widely used, liquid products have disadvantages in terms of packaging, storage, transportation, and convenience of use.

To address the disadvantages of liquid products, attempts have been made to incorporate the benefits of low Krafft point anionic surfactants into dissolvable solids. One attempt was to structure the dissolvable solid with one or more water soluble polymers via a casting and drying process. However, this process was energy intensive and costly because it involves the drying of significant amounts of water (typically >50%).

Another attempt was to create porous solids comprising low Krafft point anionic surfactants by freeze-drying. However, freeze-drying was also an energy intensive and costly process.

Producing a dissolvable personal care article using a kneader would have advantages such as eliminating the costly drying step. However, doing so is a challenge due to the hydrolytic degradation of low Krafft point anionic surfactants under high temperature kneader conditions. Additionally, low Krafft point anionic surfactants are typically available as aqueous “lamellar” pastes (comprising ˜30% water) and impart significant lubricity inside the kneader barrel which significantly limits the friction and torque between the mixing elements and the knaeder barrel, inhibiting the ability of the kneader to work effectively. Moreover, the large viscosity difference between low Krafft point anionic surfactants (as available commercially) and water soluble polymers imposes significant mixing challenges.

Based on the forgoing, there is a need for a dissolvable personal care article comprising low Krafft point anionic surfactants which can be made via a low cost kneading process.

### SUMMARY OF THE INVENTION

According to an embodiment of the invention, there is provided a process for forming a personal care article comprising (a) adding one or more water soluble polymers and one or more plasticizers to a kneader to form a premix; (b) adding one or more anionic surfactants to the premix to form a mixture; (c) kneading the mixture until homogeneous; and (d) forming the personal care article, the personal care article comprising (i) from about 10% to about 60% of one or more anionic surfactants, wherein the one or more anionic surfactants have a Krafft point of less than about 30° C.; (ii) from about 10% to about 50% of one or more water soluble polymers; (iii) from about 1% to about 30% of one or more plasticizers; and (iv) from about 10% to about 50% water.

According to another embodiment of the invention, there is provided A process for forming a personal care article comprising (a) adding one or more water soluble polymers and one or more plasticizers to a kneader; (b) heating the kneader to from about 85° C. to about 300° C. to form a premix; (c) cooling the premix to from about 80° C. to about 135° C.; (d) adding one or more anionic surfactants to the premix to form a mixture; (e) kneading the mixture until homogeneous; and (f) forming the personal care article, the personal care article comprising (i) from about 10% to about 60% of one or more anionic surfactants, wherein the one or more anionic surfactants have a Krafft point of less than about 30° C.; (b) from about 10% to about 50% of one or more water soluble polymers; (c) from about 1% to about 30% of one or more plasticizers; and (d) from about 10% to about 50% water.

These and other features, aspects, and advantages of the invention will become evident to those skilled in the art from a reading of the following disclosure.

### DETAILED DESCRIPTION OF THE INVENTION

While the specification concludes with claims which particularly point out and distinctly claim the invention, it is believed the present invention will be better understood from the following description.

In all embodiments of the present invention, all percentages are by weight of the total composition, unless specifically stated otherwise. All ratios are weight ratios, unless specifically stated otherwise. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

The term “comprising,” as used herein, means that other steps and other ingredients which do not affect the end result can be added. This term encompasses the terms “consisting of” and “consisting essentially of.” The compositions and methods/processes of the present invention can comprise, consist of, and consist essentially of the elements and limitations of the invention described herein, as well as any of the additional or optional ingredients, components, steps, or limitations described herein.

The term “kneader,” as used herein, means an intensive batch mixer. Non-limiting examples include ribbon blenders and Z-blade mixers. A benefit of this type of mixer may be the ability to sequentially add ingredients of different viscosity and achieve good blending even of materials having very different rheological profiles. The temperature may also be changed between sequential additions to accommodate materials of different thermal stability.

The term “Krafft point,” as used herein, (also known as Krafft temperature, or critical micelle temperature) means the minimum temperature at which surfactants form micelles. Below the Krafft point, there is no value for the critical micelle concentration (CMC), i.e., micelles cannot form. The Krafft point is a point of phase change below which the surfactant remains in crystalline form, even in aqueous solution. The Krafft point is measured experimentally as the temperature (more precisely, narrow temperature range) above which the solubility of a surfactant rises sharply. At this temperature, the solubility of the surfactant becomes equal to the critical micelle concentration. The Krafft point of a surfactant is best determined by locating the abrupt change in slope of a graph of the logarithm of the surfactant' s solubility versus temperature [Source: PAC, 1972, 31, 577 (Manual of Symbols and Terminology for Physicochemical Quantities and Units, Appendix II: Definitions, Terminology and Symbols in Colloid and Surface Chemistry) on page 613].

The term “plasticizer,” as used herein, means any of various substances (typically a solvent) added to a polymer composition to reduce brittleness and to promote plasticity and flexibility.

The term “semi-solid,” as used herein, means a state of matter which is highly viscous and has the qualities of both a solid and a liquid.

The term “solid,” as used herein, means a state of matter wherein the constituents are arranged such that their shape and volume are relatively stable, i.e., not liquid-like or gaseous.

The term “water soluble polymer,” as used herein, includes both water-soluble and water-dispersible polymers, and is defined as a polymer with a solubility in water, measured at 25° C., of at least about 0.1 gram/liter (g/L).

Provided is a process for forming a personal care article comprising (a) adding one or more water soluble polymers and one or more plasticizers to a kneader to form a premix; (b) adding one or more anionic surfactants to the premix to form a mixture; (c) kneading the mixture until homogeneous; and (d) forming the personal care article, the personal care article comprising (i) from about 10% to about 60% of one or more anionic surfactants, wherein the one or more anionic surfactants have a Krafft point of less than about 30° C.; (ii) from about 10% to about 50% of one or more water soluble polymers; (iii) from about 1% to about 30% of one or more plasticizers; and (iv) from about 10% to about 50% water.

### Anionic Surfactant

The personal care article may comprise from about 10% to about 60%, alternatively from about 12% to about 50%, and alternatively from about 15% to about 40% of one or more anionic surfactants, by weight of the personal care article. The one or more anionic surfactants may have a Krafft point of less than 30° C., alternatively less than 25° C., alternatively less than 20° C., alternatively less than 15° C., and alternatively less than 10° C.

Non-limiting examples of anionic surfactants may be selected from the group consisting of alkyl sulfates, alkyl ether sulfates, branched alkyl sulfates, branched alkyl alkoxylates, branched alkyl alkoxylate sulfates, alkyloxy alkane sulfonates mid-chain branched alkyl aryl sulfonates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, sodium lauroyl glutamate, and combinations thereof.

In an embodiment, the one or more anionic surfactants may comprise one or more alkyl ether sulfates according to the following structure:

[chemical structure]

wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
   wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
   wherein M+ is a monovalent counterion selected from a group consisting of sodium, potassium, ammonium, protonated monoethanolamine, protonated diethanolamine, and protonated triethanolamine; and wherein x is on average of from about 0.5 moles to about 3 moles, alternatively from about 1 mole to about 2 moles. In an embodiment, x is on average from about 0.5 moles to about 3 moles of ethylene oxide, alternatively from about 1 mole to about 2 moles of ethylene oxide.

Alkyl sulfates suitable for use herein include materials with the respective formula ROSO₃M, wherein R is an alkyl or an alkenyl of from about 8 carbon atoms to about 24 carbon atoms, and M is a water-soluble cation. Non-limiting examples of M may be selected from the group consisting of ammonium, sodium, potassium, and triethanolamine.

Non-limiting examples of alkyl ether sulfates may be selected from the group consisting of sodium laureth sulfates, ammonium laureth sulfates, potassium laureth sulfates, triethanolamine laureth sulfates, sodium trideceth sulfates, ammonium trideceth sulfates, potassium trideceth sulfates, triethanolamine trideceth sulfates, sodium undeceth sulfates, ammonium undeceth sulfates, potassium undeceth sulfates, triethanolamine undeceth sulfates, and combinations thereof. In an embodiment, the alkyl ether sulfate may be sodium laureth sulfates.

Other suitable anionic surfactants may be described in McCutcheon's Detergents and Emulsifiers, North American Edition (1986), Allured Publishing Corp.; McCutcheon's Functional Materials, North American Edition (1992), Allured Publishing Corp; and U.S. Pat. Nos. 2,486,921, 2,486,922, and 2,396,278.

### Secondary Surfactant

The personal care article may further comprise one or more secondary surfactants selected from the group consisting of amphoteric surfactants, zwitterionic surfactants, and mixtures thereof. The ratio of the one or more anionic surfactants to the one or more secondary surfactants may be from about 15:1 to about 1:2, alternatively from about 10:1 to about 1:1.

Non-limiting examples of amphoteric surfactants may be selected from the group consisting of aliphatic derivatives of secondary and tertiary amines, aliphatic derivatives of heterocyclic secondary and tertiary amines, and mixtures thereof.

Further non-limiting examples of amphoteric surfactants may be selected from the group consisting of sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethy-7 carboxyamphodiacetate, lauraminopropionic acid, lauroamphodipropionic acid, lauryl aminopropylglycine, lauryl diethylenediaminoglycine, and mixtures thereof.

Non-limiting examples of zwitterionic surfactants may be selected from the group consisting of derivatives of secondary and tertiary amines, derivatives of heterocyclic secondary and tertiary amines, derivatives of quaternary ammonium, derivatives of quaternary phosphonium, derivatives of tertiary sulfonium, and mixtures thereof.

Non-limiting examples of zwitterionic surfactants may also be selected from the group consisting of betains including alkyl dimethyl betaine and cocodimethyl amidopropyl betaine, C₈-C₁₈ amine oxides, sulfo and hydroxy betaines, and mixtures thereof.

Further non-limiting examples of zwitterionic surfactants may be selected from the group consisting of cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, oleamidopropyl betaine, coco-sultaine, lauramidopropyl betaine, lauryl betaine, lauryl hydroxysultaine, lauryl sultaine, and mixtures thereof.

### Water-Soluble Polymer

The personal care article may comprise one or more water soluble polymers that may function as a structurant. The personal care article may comprise from about 10% to about 50%, alternatively from about 15% to about 45%, alternatively from about 20% to about 40%, and alternatively from about 25% to about 35% of one or more water soluble polymers, by weight of the personal care article.

The one or more water soluble polymers may have solubility in water, measured at 25° C., of from about 0.1 g/L to about 500 g/L. The one or more water soluble polymers may be of synthetic or natural origin and may be modified by means of a chemical reaction.

In an embodiment, the one or more water soluble polymers may have a weight average molecular weight of from about 40,000 g/mol to about 500,000 g/mol, alternatively from about 50,000 g/mol to about 400,000 g/mol, alternatively from about 60,000 g/mol to about 300,000 g/mol, and alternatively from about 70,000 g/mol to about 200,000 g/mol.

In an embodiment, a 4% by weight solution of one or more water soluble polymers may have a viscosity at 20° C. of from about 4 centipoise to about 80 centipoise, alternatively from about 10 centipoise to about 60 centipoise, and alternatively from about 20 centipoise to about 40 centipoise.

Non-limiting examples of synthetic water soluble polymers may be selected from the group consisting of polyvinyl alcohols, polyvinylpyrrolidones, polyalkylene oxides, polyacrylates, caprolactams, polymethacrylates, polymethylmethacrylates, polyacrylamides, polymethylacrylamides, polydimethylacrylamides, polyethylene glycol monomethacrylates, polyurethanes, polycarboxylic acids, polyvinyl acetates, polyesters, polyamides, polyamines, polyethyleneimines. Further non-limiting examples of synthetic water soluble polymers may be selected from the group consisting of copolymers of anionic, cationic and amphoteric monomers and mixtures thereof, including maleic acrylate based copolymers, maleic methacrylate based copolymers, copolymers of methylvinyl ether and of maleic anhydride, copolymers of vinyl acetate and of crotonic acid, copolymers of vinylpyrrolidone and of vinyl acetate, and copolymers of vinylpyrrolidone and of caprolactam.

Non-limiting examples of natural water soluble polymers may be selected from the group consisting of karaya gum, tragacanth gum, gum arabic, acemannan, konjac mannan, acacia gum, gum ghatti, whey protein isolate, soy protein isolate, guar gum, locust bean gum, quince seed gum, psyllium seed gum, carrageenan, alginates, agar, fruit extracts (pectins), xanthan gum, gellan gum, pullulan, hyaluronic acid, chondroitin sulfate, and dextran, casein, gelatin, keratin, keratin hydrolysates, sulfonic keratins, albumin, collagen, glutelin, glucagons, gluten, zein, shellac, and mixtures thereof.

Non-limiting examples of modified natural water soluble polymers may be selected from the group consisting of (1) cellulose derivatives including hydroxypropylmethylcellulose, hydroxymethylcellulose, hydroxyethylcellulose, methylcellulose, hydroxypropylcellulose, ethylcellulose, carboxymethylcellulose, cellulose acetate phthalate, nitrocellulose, cellulose ethers, cellulose esters; and (2) guar derivatives including hydroxypropyl guar. Suitable hydroxypropylmethylcelluloses may include those available from the Dow Chemical Company (Midland, Mich.).

In an embodiment, the one or more water soluble polymers may be blended with a starch-based material in such an amount as to reduce the overall level of water soluble polymer required. The combined weight percentage of the one or more water soluble polymers and the starch-based material may range from about 10% to about 40%, alternatively from about 12% to about 30%, and alternatively from about 15% to about 25%, by weight of the personal care article. The weight ratio of the one or more water soluble polymers to the starch-based material may range from about 1:10 to about 10:1, alternatively from about 1:8 to about 8:1, alternatively from about 1:7 to about 7:1, and alternatively from about 6:1 to about 1:6.

Non-limiting examples of starch-based materials may be selected from the group consisting of cereals, tubers, roots, legumes, fruits, and combinations thereof. More specifically, non-limiting examples of starch-based materials may be selected from the group consisting of corn, peas, potatoes, bananas, barley, wheat, rice, sago, amaranth, tapioca, arrowroot, canna, sorghum, and combinations thereof. The starch-based materials may also include native starches that are modified using any modification known in the art, including physically modified starches and chemically modified starches.

### Plasticizer

The personal care article may comprise one or more plasticizers. The personal care article may comprise from about 1% to about 30%, alternatively from about 5% to about 25%, and alternatively from about 10% to about 20% of one or more plasticizers, by weight of the personal care article. Non-limiting examples of plasticizers may be selected from the group consisting of polyols, copolyols, polycarboxylic acids, polyesters, dimethicone copolyols, and mixtures thereof.

Non-limiting examples of suitable polyols may be selected from the group consisting of glycerin, diglycerin, propylene glycol, ethylene glycol, butylene glycol, pentylene glycol, cyclohexane dimethanol, hexanediol, polyethylene glycol, sorbitol, manitol, lactitol, monohydric and polyhydric low molecular weight alcohols (e.g., C₂-C₈ alcohols), monosaccharides, disaccharides, oligosaccharides, high fructose corn syrup solids, ascorbic acid, and mixtures thereof.

Non-limiting examples of suitable polycarboxylic acids may be selected from the group consisting of citric acid, maleic acid, succinic acid, polyacrylic acid, polymaleic acid, and mixtures thereof.

Non-limiting examples of suitable polyesters may be selected from the group consisting of glycerol triacetate, acetylated-monoglyceride, diethyl phthalate, triethyl citrate, tributyl citrate, acetyl triethyl citrate, acetyl tributyl citrate, and mixtures thereof.

Non-limiting examples of suitable dimethicone copolyols may be selected from the group consisting of PEG-12 dimethicone, PEG/PPG-18/18 dimethicone, and PPG-12 dimethicone.

Further non-limiting examples of suitable plasticizers may be selected from the group consisting of alkyl phthalates, allyl phthalates, napthalates, lactates (e.g., sodium, ammonium and potassium salts), sorbeth-30, urea, lactic acid, sodium pyrrolidone carboxylic acid (PCA), sodium hyaluronate, hyaluronic acid, soluble collagen, modified protein, monosodium L-glutamate, glyceryl polymethacrylate, polymeric plasticizers, proteins, amino acids, hydrogen starch hydrolysates, low molecular weight esters (e.g., esters of C₂-C₁₀ alcohols and acids), and mixtures thereof. In an additional embodiment, non-limiting examples of suitable plasticizers may be alpha and beta hydroxyl acids selected from the group consisting of glycolic acid, lactic acid, citric acid, maleic acid, salicylic acid, and mixtures thereof. EP 0283165 B1 discloses even more suitable plasticizers, including glycerol derivatives such as propoxylated glycerol.

### Water

The personal care article may comprise from about 10% to about 50%, alternatively from about 15% to about 45%, alternatively from about 20% to about 40% water, by weight of the personal care article.

### Benefit Agent

The personal care article may comprise from about 0.1% to about 15% of a benefit agent. Non-limiting examples of suitable benefit agents may be selected from the group consisting of nonionic surfactants, preservatives, perfumes, coloring agents, cationic polymers, conditioning agents, hair bleaching agents, thickeners, moisturizers, emollients, pharmaceutical actives, vitamins, sunscreens, deodorants, sensates, plant extracts, cosmetic particles, reactive agents, skin lightening agents, skin tanning agents, anti-dandruff agents, exfoliating agents, acids, bases, humectants, enzymes, suspending agents, pH modifiers, hair perming agents, anti-acne agents, anti-microbial agents, exfoliation particles, hair growth agents, insect repellents, chelants, dissolution aids, builders, enzymes, dye transfer inhibiting agents, softening agents, and mixtures thereof.

In an embodiment, the personal care article may be configured as a lubricating strip on a disposable shaving device.

### Conditioning Agents

Non-limiting examples of conditioning agents may be selected from the group consisting of silicones, organic oils,and mixtures thereof. Non-limiting examples of silicones may be selected from the group consisting of silicone oils, high molecular weight polyalkyl or polyaryl siloxanes, aminosilicones, cationic silicones, silicone gums, high refractive silicones, low molecular weight polydimethyl siloxanes, silicone resins, and mixtures thereof. Non-limiting examples of organic oils may be selected from the group consisting of hydrocarbon oils, polyolefins, fatty esters, and mixtures thereof. Additional non-limiting examples of conditioning agents and optional suspending agents for silicone may be found in U.S. Pat. Nos. 5,104,646 and 5,106,609, which are incorporated herein by reference.

The silicone gums and the high molecular weight polyalkyl or polyaryl siloxanes may have a viscosity of from about 100,000 mPa·s to about 30,000,000 mPa·s, alternatively from about 200,000 mPa·s to about 30,000,000 mPa·s. The silicone gums and the high molecular weight polyalkyl or polyaryl siloxanes may have a molecular weight of from about 100,000 g/mol to about 1,000,000 g/mol, and alternatively from about 120,000 g/mol to about 1,000,000 g/mol.

The low molecular weight polydimethyl siloxanes may have a viscosity of from about 1 mPa·s to about 10,000 mPa·s at 25° C., and alternatively from about 5 mPa·s to about 5,000 mPa·s. The low molecular weight polydimethyl siloxanes may have a molecular weight of from about 400 to about 65,000, and alternatively from about 800 to about 50,000.

In an embodiment, the conditioning agent may include one or more aminosilicones. Aminosilicones may be silicones containing at least one primary amine, secondary amine, tertiary amine, or a quaternary ammonium group. In an embodiment the aminosilicones may have less than about 0.5% nitrogen by weight of the aminosilicone, in another embodiment less than about 0.2%, in yet another embodiment less than about 0.1%.

In an embodiment, the aminosilicones may have a particle size of less than about 50 m once incorporated into the final composition. The particle size measurement may be taken from dispersed droplets in the final composition. Particle size may be measured by means of a laser light scattering technique using a Horiba model LA-910 Laser Scattering Particle Size Distribution Analyzer (Horiba Instruments, Inc.).

The aminosilicones may have a viscosity of from about 1,000 cs (centistokes) to about 1,000,000 cs, in another embodiment from about 10,000 cs to about 700,000 cs, in yet another embodiment from about 50,000 cs to about 500,000 cs, and in yet another embodiment from about 100,000 cs to about 400,000 cs. This embodiment may also comprise a low viscosity fluid. The viscosity of aminosilicones discussed herein is measured at 25° C.

In another embodiment, the aminosilicones may have a viscosity of from about 1,000 cs to about 100,000 cs, in another embodiment from about 2,000 cs to about 50,000 cs, in another embodiment from about 4,000 cs to about 40,000 cs, and in yet another embodiment from about 6,000 cs to about 30,000 cs.

The personal care composition may comprise from about 0.05% to about 20%, alternatively from about 0.1% to about 10%, and alternatively from about 0.3% to about 5% aminosilicones by weight of the personal care composition.

### Anti-Dandruff Agents

In an embodiment, the personal care article may comprise an anti-dandruff agent which may be an anti-dandruff particulate. Non-limiting examples of suitable anti-dandruff agents may be selected from the group consisting of pyridinethione salts, azoles (e.g. ketoconazole, econazole, and elubiol), selenium sulphide, particulate sulfur, keratolytic agents (e.g. salicylic acid), and mixtures thereof. In an embodiment, the anti-dandruff agent is a pyridinethione salt.

Pyridinethione salt particulates are suitable particulate anti-dandruff agents. In an embodiment, the anti-dandruff agent may be a 1-hydroxy-2-pyridinethione salt in particulate form. The personal care article may comprise from about 0.01% to about 5%, alternatively from about 0.1% to about 3%, and alternatively from about 0.1% to about 2% pyridinethione salt particulates. In an embodiment, the pyridinethione salt particulates may be those formed from heavy metals such as zinc, tin, cadmium, magnesium, aluminium, and zirconium. In any embodiment, the pyridinethione salt may be the zinc salt of 1-hydroxy-2-pyridinethione (known as “zinc pyridinethione” or “ZPT”) optionally in platelet particle form. In an embodiment, the zinc salt of 1-hydroxy-2-pyridinethione in platelet particle form may have an average particle size of less than 20 microns, alternatively less than 5 microns, and alternatively less than 2.5 microns. Salts formed from other cations, such as sodium, may also be suitable anti-dandruff agents. Pyridinethione anti-dandruff agents are described, for example, in U.S. Pat. Nos. 4,323,683; 4,379,753; and 4,470,982.

The personal care article may also comprise an antimicrobial active. Non-limiting examples of suitable anti-microbial actives may be selected from the group consisting of coal tar, sulfur, charcoal, aluminum chloride, gentian violet, octopirox (piroctone olamine), ciclopirox olamine, undecylenic acid and its metal salts, potassium permanganate, selenium sulphide, sodium thiosulfate, propylene glycol, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, azoles, and mixtures thereof. Further non-limiting examples of suitable anti-microbial agents may be selected from the group consisting of itraconazole, ketoconazole, selenium sulphide, coal tar, and mixtures thereof.

In an embodiment, the anti-microbial agent may be an imidazole selected from the group consisting of benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof. In an embodiment, the anti-microbial agent may be a triazole selected from the group consisting of terconazole, itraconazole, and mixtures thereof.

### Cationic Polymers

In an embodiment, the personal care article may comprise a cationic polymer. Cationic polymers useful herein may include those discussed in US 2007/0207109 A1 and US 2008/0206185 A1, such as synthetic copolymers of sufficiently high molecular weight to effectively enhance the deposition of the conditioning active components of the personal care article described herein. Combinations of cationic polymer may also be utilized. The average molecular weight of the synthetic copolymers is generally between about 10,000 and about 10 million, preferably between about 100,000 and about 3 million, still more preferably between about 200,000 and about 2 million.

In a further embodiment, the synthetic copolymers have mass charge densities of from about 0.1 meq/gm to about 6.0 meq/gm, alternatively from about 0.5 meq/gm to about 3.0 meq/gm, at the pH of intended use of the personal care article. The pH may be from about pH 3 to about pH 9, and alternatively from about pH 4 and about pH 8.

In yet another embodiment, the synthetic copolymers have linear charge densities from at least about 2 meq/A to about 500 meq/A, and more preferably from about 20 meq/A to about 200 meq/A, and most preferably from about 25 meq/A to about 100 meq/A.

Cationic polymer may be copolymers or homopolymers. In one embodiment, a homopolymer is utilized in the present composition. In another embodiment, a copolymer is utilized in the present composition. In another embodiment a mixture of a homopolymer and a copolymer is utilized in the present composition. In another embodiment, a homopolymer of a naturally derived nature, such as cellulose or guar polymer discussed herein, is combined with a homopolymer or copolymer of synthetic origin, such as those discussed below.

Homopolymers—Non-crosslinked cationic homopolymers of the following monomers are also useful herein: 3-acrylamidopropyltrimethylammonium chloride (APTAC), diallyldimethylammonium chloride (DADMAC), [(3-methylacrylolyamino)propyl]trimethylammonium chloride (MAPTAC), 3-methyl-1-vinylimidazolium chloride (QVI); [2-(acryloyloxy)ethyl]trimethylammonium chloride and [2-(acryloyloxy)propyl]trimethylammonium chloride.

Copolymers—copolymer may be comprises of two cationic monomer or a nonionic and cationic monomers.

The personal care articles may also comprise cellulose or guar cationic deposition polymers. Generally, such cellulose or guar cationic deposition polymers may be present at a concentration from about 0.05% to about 5%, by weight of the composition. Suitable cellulose or guar cationic deposition polymers have a molecular weight of greater than about 5,000. Additionally, such cellulose or guar deposition polymers have a charge density from about 0.5 meq/g to about 4.0 meq/g at the pH of intended use of the personal care article, which pH will generally range from about pH 3 to about pH 9, preferably between about pH 4 and about pH 8. The pH of the compositions is measured neat.

In one embodiment of the invention, the cationic polymers are derivatives of Hydroxypropyl Guar, examples of which include polymers known via the INCI nomenclature as Guar Hydroxypropyltrimonium Chloride, such as the products sold under the name Catinal CG-100, Catinal CG-200 by the company Toho, Cosmedia Guar C-261N, Cosmedia Guar C-261N, Cosmedia Guar C-261N by the company Cognis, DiaGum P 5070 by the company Freedom Chemical Diamalt, N-Hance Cationic Guar by the company Hercules/Aqualon, Hi-Care 1000, Jaguar C-17, Jaguar C-2000, Jaguar C-13S, Jaguar C-14S, Jaguar Excel by the company Rhodia, Kiprogum CW, Kiprogum NGK by the company Nippon Starch.

### Process of Forming the Personal Care Article

The process of forming a personal care article may comprise (a) adding one or more water soluble polymers and one or more plasticizers to a kneader to form a premix; (b) heating the premix to from about 80° C. to about 135° C.; (c) adding one or more anionic surfactants to the premix to form a mixture; (d) kneading the mixture until homogeneous; and (e) forming the personal care article, the personal care article comprising (i) from about 10% to about 60% of one or more anionic surfactants, wherein the one or more anionic surfactants have a Krafft point of less than about 30° C.; (ii) from about 10% to about 50% of one or more water soluble polymers; (iii) from about 1% to about 30% of one or more plasticizers; and (iv) from about 10% to about 50% water.

In an embodiment, the premix may be heated to a temperature of from about 85° C. to about 300° C. before bringing the temperature of the premix to from about 80° C. to about 135° C.

### EXAMPLE 1

| Component | Wt. % |
|---|---|
| Additional Water | 31.90% |
| Polyvinyl Alcohol | 26.42% |
| Cationic Polymer | 0.63% |
| Citric Acid | 0.48% |
| Glycerin | 4.41% |
| Sodium Laureth 1 Sulfate (70% active) | 19.77% |
| Sodium Laureth 3 Sulfate (28% Active) | 6.90% |
| Sodium Lauroamphoacetate (80% Active) | 3.83% |
| Perfume | 1.81% |
| Amino Silicone Fluid | 3.85% |

In this example, a kneader is heated to about 95° C. Water, polyvinyl alcohol, cationic polymer, and citric acid are added and are mixed for about 30 minutes. Mixing is stopped, and glycerin, SLE1S, SLE2S, NaLAA are added. Mixing is resumed for about 30 minutes. Mixing is stopped again, and perfume and amino silicone fluid are added. Mixing is resumed for about 15 minutes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited herein are incorporated herein by reference in their entirety; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for forming a personal care article comprising:
a. adding one or more water soluble polymers and one or more plasticizers to a kneader to form a premix;
b. adding one or more anionic surfactants to the premix to form a mixture;
c. kneading the mixture until homogeneous; and
d. forming the personal care article, the personal care article comprising:
i. from about 10% to about 60% of one or more anionic surfactants, by weight of the personal care article, wherein the one or more anionic surfactants have a Krafft point of less than about 30° C.;
ii. from about 10% to about 50% of one or more water soluble polymers, by weight of the personal care article;
iii. from about 1% to about 30% of one or more plasticizers, by weight of the personal care article; and
iv. from about 10% to about 50% water, by weight of the personal care article.

2. The process of claim 1, wherein the one or more anionic surfactants have a Krafft point of less than about 25° C.

3. The process of claim 1, wherein the one or more anionic surfactants comprises one or more alkyl ether sulfates according to the following structure: [chemical structure]
wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
wherein M+ is a monovalent counterion selected from a group consisting of sodium, potassium, ammonium, protonated monoethanolamine, protonated diethanolamine, and protonated triethanolamine; and
wherein x is on average from about 0.5 moles to about 3 moles.

4. The process of claim 3, wherein x is on average from about 0.5 moles to about 3.0 moles of ethylene oxide.

5. The process of claim 3, wherein x is on average from about 1.0 mole to about 2.0 moles of ethylene oxide.

6. The process of claim 3, wherein the alkyl ether sulfate is sodium laureth sulfate.

7. The process of claim 1, wherein the personal care article comprises from about 15% to about 50% of one or more anionic surfactants.

8. The process of claim 1, wherein the personal care article comprises from about 20% to about 40% of the one or more anionic surfactants.

9. The process of claim 1, wherein the personal care article comprises from about 15% to about 45% water.

10. The process of claim 1, wherein the personal care article comprises from about 20% to about 40% water.

11. The process of claim 1, wherein the one or more water soluble polymers is selected from the group consisting of polyvinyl alcohol, polyvinylpyrrolidone, polyalkylene oxide, starch, starch derivatives, pullulan, gelatin, hydroxypropylmethylcellulose, methycellulose, carboxymethycellulose, and mixtures thereof.

12. The process of claim 1, wherein the one or more water soluble polymers is polyvinyl alcohol.

13. The process of claim 1, wherein the one or more plasticizers is selected from the group consisting of glycerin, propylene glycol, polyols, copolyols, polycarboxylic acids, polyesters, dimethicone copolyols, and mixtures thereof.

14. The process of claim 1, wherein the personal care article further comprises a secondary surfactant selected from the group consisting of amphoteric surfactants, zwitterionic surfactants, and mixtures thereof; and wherein the ratio of the one or more anionic surfactants to the secondary surfactant is from about 10:1 to about 1:2.

15. The process of claim 1, wherein the personal care article further comprises from about 0.1% to about 15% of one or more benefit agents.

16. The process of claim 15, wherein the one or more benefit agents are selected from the group consisting of anti-dandruff agents, conditioning agents, moisturizers, and combinations thereof.

17. The process of claim 16, wherein the conditioning agent is selected from the group consisting of silicones, aminosilicones, quaternized silicones, and combinations thereof.

18. The process of claim 1, wherein the personal care article further comprises a cationic polymer.

19. A process for forming a personal care article comprising:
a. adding one or more water soluble polymers and one or more plasticizers to a kneader;
b. heating the premix to from about 80° C. to about 135° C.;
c. adding one or more anionic surfactants to the premix to form a mixture;
d. kneading the mixture until homogeneous; and
e. forming the personal care article, the personal care article comprising:
i. from about 10% to about 60% of one or more anionic surfactants, by weight of the personal care article, wherein the one or more anionic surfactants have a Krafft point of less than about 30° C.;
ii. from about 10% to about 50% of one or more water soluble polymers, by weight of the personal care article;
iii. from about 1% to about 30% of one or more plasticizers, by weight of the personal care article; and
iv. from about 10% to about 50% water, by weight of the personal care article.

20. The process of claim 19, wherein the one or more anionic surfactants comprises an alkyl ether sulfate according to the following structure: [chemical structure]
wherein R¹ is a C-linked monovalent substituent selected from the group consisting of:
a. substituted alkyl systems comprising from about 9 to about 15 carbon atoms;
b. unsubstituted alkyl systems comprising from about 9 to about 15 carbon atoms;
c. straight alkyl systems comprising from about 9 to about 15 carbon atoms;
d. branched alkyl systems comprising from about 9 to about 15 carbon atoms; and
e. unsaturated alkyl systems comprising from about 9 to about 15 carbon atoms;
wherein R² is selected from the group consisting of:
a. C-linked divalent straight alkyl systems comprising from about 2 to about 3 carbon atoms;
b. C-linked divalent branched alkyl systems comprising from about 2 to about 3 carbon atoms; and
c. combinations thereof;
wherein M+ is a monovalent counterion selected from a group consisting of sodium, potassium, ammonium, protonated monoethanolamine, protonated diethanolamine, and protonated triethanolamine; and
wherein x is on average from about 0.5 moles to about 3 moles.
